(19) United States
(12) Patent Application Publication
(10) Pub. No.: US 2013/0309140 A1
(43) Pub. Date: Nov. 21, 2013

(54) CORONA DISCHARGE OZONE GENERATOR

(71) Applicant: JOHN P. BARRETT, WOODSTOCK, IL (US)

(72) Inventor: JOHN P. BARRETT, WOODSTOCK, IL (US)

(21) Appl. No.: 13/896,199

(22) Filed: May 16, 2013

Related U.S. Application Data
(60) Provisional application No. 61/688,568, filed on May 17, 2012.

(51) Int. Cl. A61L 9/015

(52) U.S. Cl. CPC A61L 9/015
USPC 422/124

(57) ABSTRACT

The corona discharge ozone generator is a portable ozone generator designed to produce a sufficient amount of ozone to kill, inactivate or suppress noxious odors, mold, mildew, bacteria, fungus, pathogens, small pests and the like. The generator includes a housing enclosing a ceramic dielectric plate having at least two spaced electrodes. A module for generating a high voltage and low current is also enclosed in the housing. The module applies a high voltage and low current across the electrodes to produce a corona discharge between the surfaces of the electrodes. The discharge causes ozone to be produced in ambient air. The ceramic plate enhances the ozone production. A fan is mounted on or in the housing to disperse the ozone as needed. Ozone is not stored within the device. The module can be powered by AC, DC, solar or other conventional sources. An on/off switch is mounted on the housing for activating and deactivating the module.

### CROSS-REFERENCE TO RELATED APPLICATION

This application claims the benefit of U.S. Provisional Patent Application Ser. No. 61/688,568, filed May 17, 2012.

### BACKGROUND OF THE INVENTION

1. Field of the Invention

The present invention generally relates to air purifiers and devices for controlling airborne pathogens, and particularly to a corona discharge ozone generator that is used to eliminate odors and render airborne pathogens harmless.

2. Description of the Related Art

Offensive odors and small pests are two maladies that plague residential, commercial and recreational environments. Offensive odors usually occur in enclosed spaces and may develop from many sources. Pets, mold, cooking, dampness, cigarettes, and the like, are just a few of the sources that may give rise to offensive odors in a home, office, classroom, gymnasium, automobile and the like. Many diseases are cause by microbes carried by bacteria, viruses, fungi, mildew and the like that are spread by spores or by small insect pests, such as mites, gnats, flies, and the like. Efforts to eliminate offensive odors and small pests have spawned a lucrative industry that is involved in the production and marketing of aromatic candles, fragrant sprays, evaporative scented liquids and the like to allay the odors. Pesticides, traps, and stored toxic materials are employed to eliminate the pests. Most of the odor-allaying products function only to mask the offensive odors, replacing the same with an ostensibly more pleasant aroma. Unfortunately the pleasant aroma is often excessively flowery or antiseptic, creating a less-than-adequate aura.

Pesticides are dangerous to humans, and especially to children, if accidently ingested or inhaled to excess. Disposing of pest-filled traps and storage of toxic materials is an unpleasant task and a nuisance that can be very costly to both ship and store. An unobtrusive device that can rid the ambient air in a space of noxious odors, pests and leave a clean, natural smell would certainly be a welcome addition to the art. Thus, a relatively small, hand-held corona discharge ozone generator solving the aforementioned problems is desired.

### SUMMARY OF THE INVENTION

The corona discharge ozone generator is a portable, relatively small, hand-held ozone generator designed to produce a sufficient amount of ozone to eliminate, kill, inactivate or suppress noxious odors, mold, mildew, bacteria, fungus, pathogens, small pests and the like. The generator comprises a housing enclosing a ceramic dielectric plate having at least two spaced electrodes (anode and cathode). A module for generating a high voltage and low current is also enclosed in the housing. The module applies a high voltage and low current across the electrodes to produce a corona discharge between the surfaces of the electrodes. The discharge causes ozone to be produced in ambient air. The ceramic plate enhances the ozone production. A fan is mounted in or on the housing to disperse the produced ozone as generated. There is no ozone stored in the housing. The module can be powered by AC, DC, solar or most conventional power sources. An on/off switch is mounted on the housing for activating and deactivating the module.

Accordingly, the invention presents a device for producing ozone that is durable, efficient and easy to use. The device is portable and does not store unused ozone therein. The invention provides for improved elements thereof in an arrangement for the purposes described that are inexpensive, dependable and fully effective in accomplishing their intended purposes.

These and other features of the present invention will become readily apparent upon further review of the following specification and drawings.

### BRIEF DESCRIPTION OF THE DRAWINGS

FIG. 1 is an environmental perspective view of a first embodiment of a corona discharge ozone generator according to the present invention.

FIG. 2 is a rear view of the corona discharge ozone generator of FIG. 1 according to the present invention.

FIG. 3 is a side view of the corona discharge ozone generator of FIG. 1 according to the present invention.

FIG. 4 is a bottom view of the corona discharge ozone generator of FIG. 1 according to the present invention.

FIG. 5 is a side view in section of the corona discharge ozone generator of FIG. 1 according to the present invention, showing the power module and ceramic ozone producing plate.

FIG. 6 is an environmental perspective view of a second embodiment of a corona discharge ozone generator according to the present invention.

FIG. 7 is a side view in section of the corona discharge ozone generator of FIG. 6 according to the present invention, showing the power module, ceramic ozone producing plate and fan placement.

Similar reference characters denote corresponding features consistently throughout the attached drawings.

### DETAILED DESCRIPTION OF THE PREFERRED EMBODIMENT

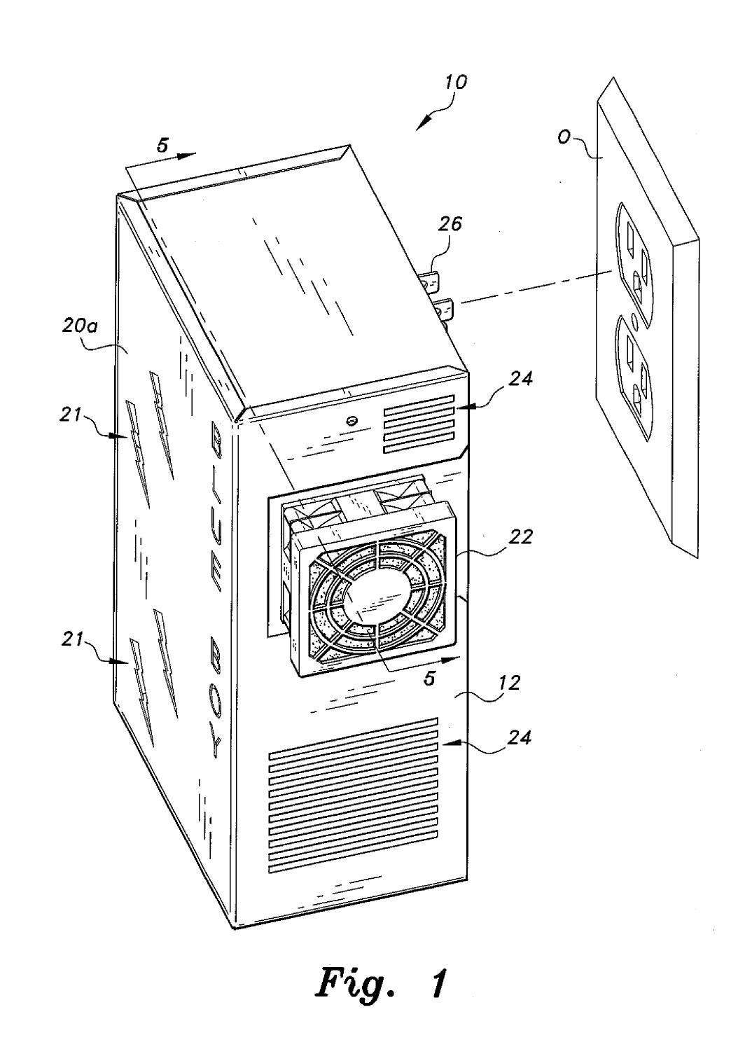

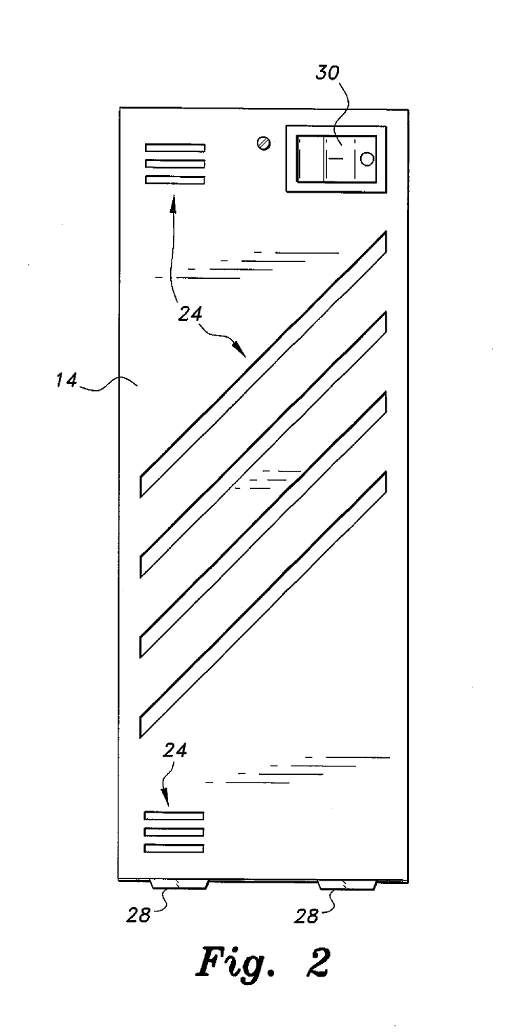

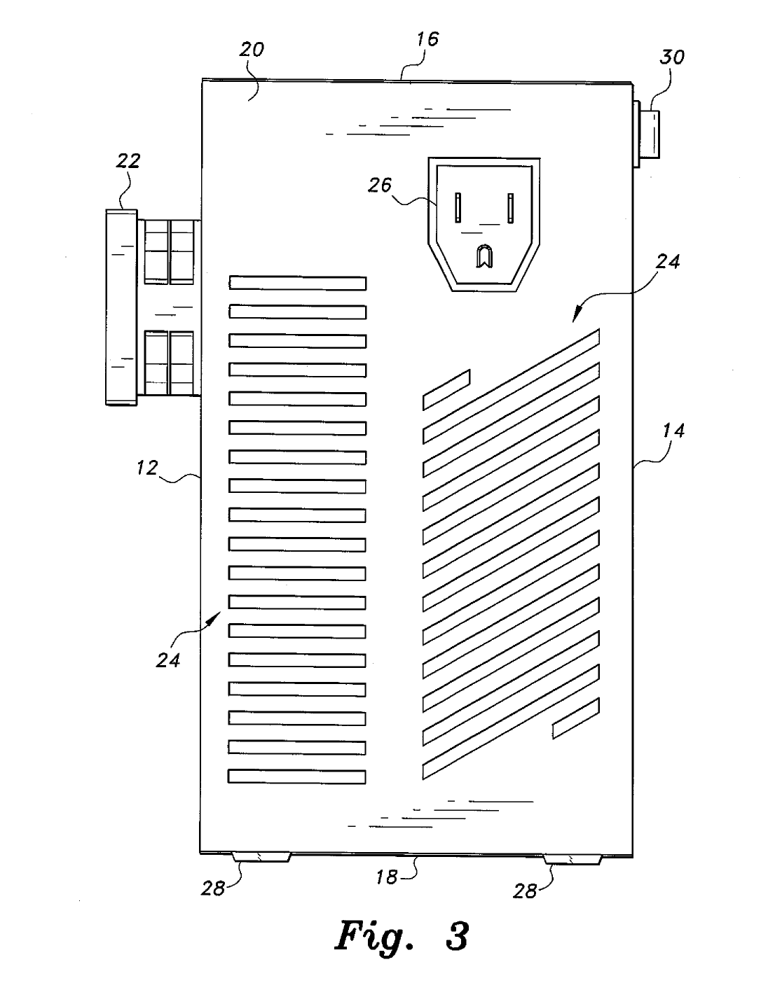

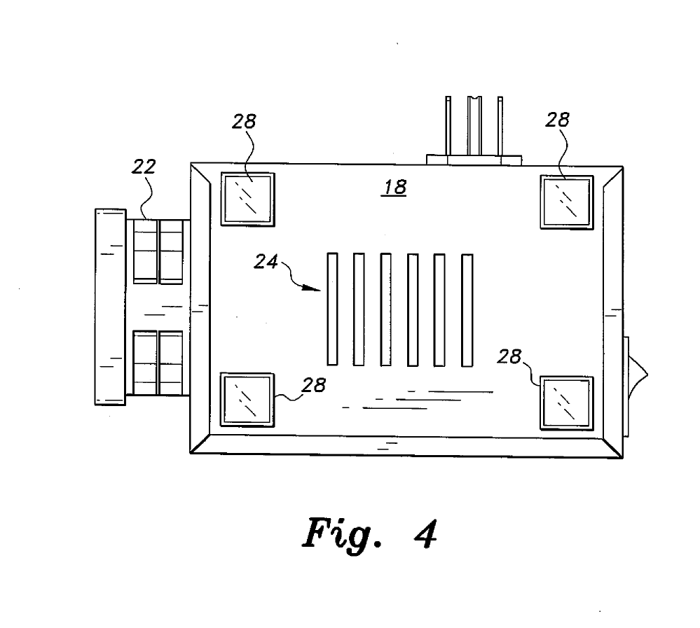

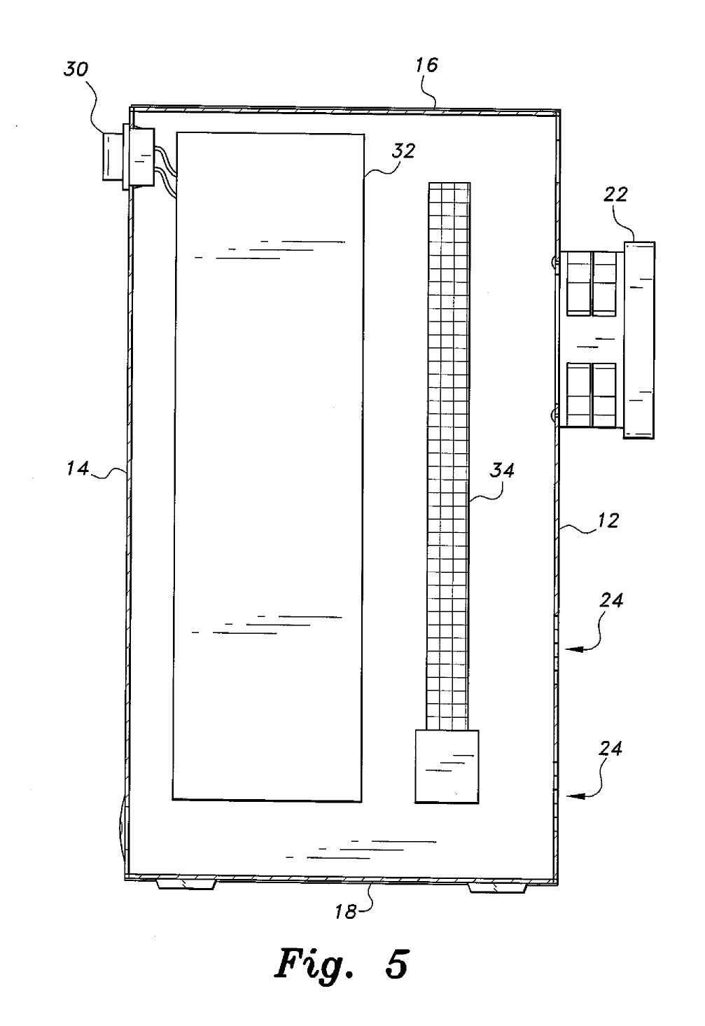

Referring to FIGS. 1-5, the corona discharge ozone generator is generally indicated at 10. The generator 10 comprises a housing having front wall 12, a rear wall 14, a top wall 16, a bottom wall 18, and opposing sidewalls 20, 20a. The walls are attached to enclose a space therein for containing apparatus for the generation and distribution of ozone, as will be further explained below. The walls can be fabricated from any suitable material that is resistant to oxidation. A fan 22 is mounted in the front wall 12 for drawing ambient air into the housing. It should be noted that the fan can be mounted on any suitable wall. Openings in the form of vents or slots 24 are mounted in the front, rear, bottom and sidewalls to allow dispensing of ozone from the space defined by the housing. An electrical plug 26 is mounted on one of the sidewalls 20 for connecting the device to a source of AC power at outlet 0. Optionally, the device may be powered by batteries, which may be rechargeable or may be replaceable dry-call batteries. The sidewall 20a is formed with openings 21 (or slots) therein for exposing LED lights (not shown). Feet 28, preferably fabricated from nylon, are disposed on the bottom wall 18 to prevent the generator from marring any surface on which it may sit. Optionally, feet may be installed on a suitable sidewall. An on/off switch 30, whose function is explained below, is disposed on the rear wall 14. A high-voltage electric power supply module 32 is positioned in the space defined by the walls of the housing. A dielectric, ceramic plate 34 having at least two spaced electrodes is positioned adjacent to the high-voltage power supply module 32. The fan 22 and the ceramic plate 34 are electrically wired (not shown) to the power supply module 32 in a conventional manner.

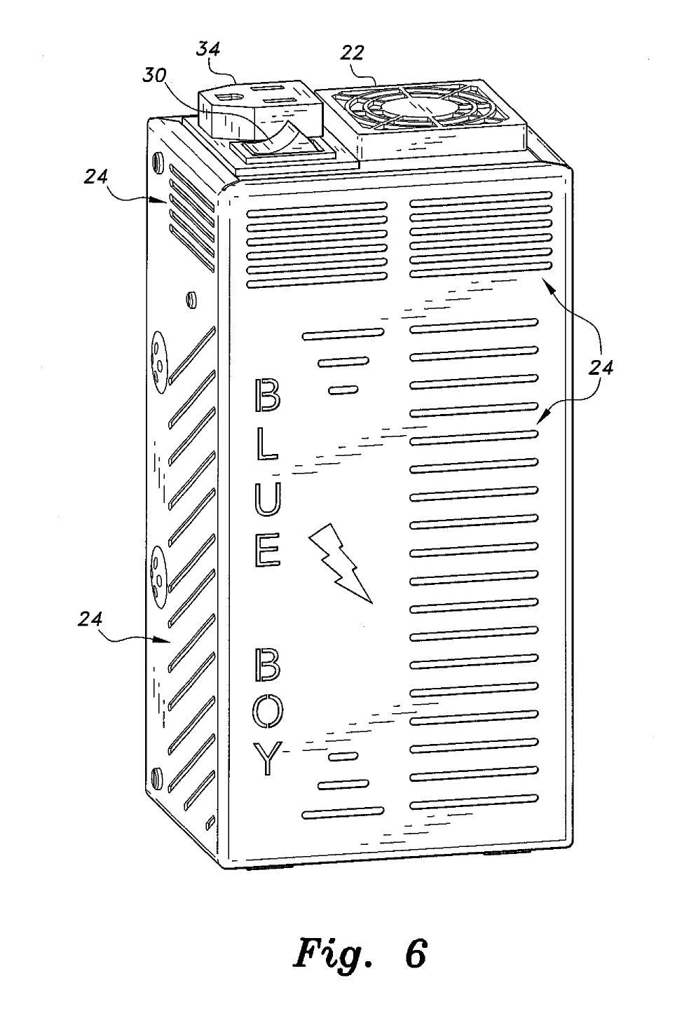

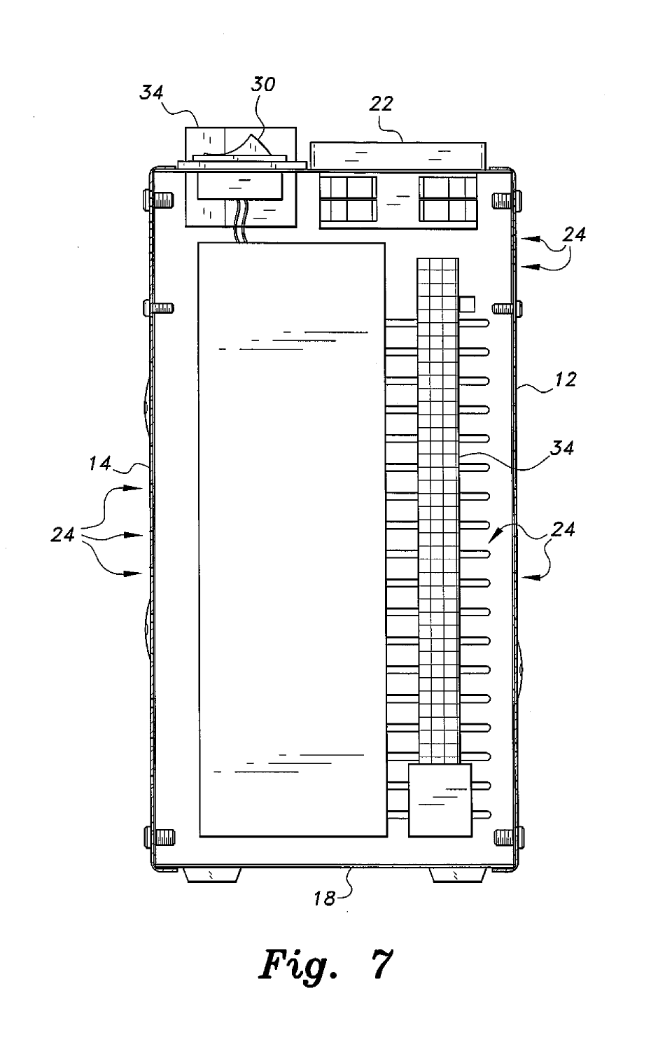

FIGS. 6 and 7 show a second embodiment of the invention wherein the fan is encased in the housing adjacent the top wall 16 thereof. An AC jack 34 and switch 30 combination are positioned adjacent the fan inlet. This embodiment allows the use of AC devices such as power cords, timers, remote control devices and the like.

In use, the generator is connected to an outside power source (AC, DC, solar, and the like.). The on/off switch 30 is manipulated to activate the module 32 and fan 22. Ambient air is drawn into the generator via fan 22. Power supplied to the ceramic plate creates ozone as described above. The fan blows air over the dielectric plate to strip ozone from the plate. The ozone is dispersed to a finite surrounding area via the slots 24. The air flow generated by the fan 22 will also disperse heat caused by the electronic components including the ceramic plate and power source. The dispersed ozone will effectively rid the finite area of odors, mold, mildew, bacteria, fungus, pathogens, small pests, and the like.

Ozone (O₃) is a chemical containing three atoms of oxygen per molecule. It is much less stable than the diatomic form of oxygen (O₂), and is a powerful oxidant. Ozone is toxic to many forms of bacteria, viruses, fungi, and other airborne microorganisms. Ozone is also very reactive with many substances that cause odor, oxidizing these substances to forms that are less odiferous. For this reason, ozone is often used as a sanitizer to rid areas of noxious odors and harmful microbes or microorganisms.

It is recommended that people and pets evacuate the area for a time period (approximately 1.5 hours total) during and after ozone treatment. It is also recommended that plants and small electronic devices be removed from the finite space surrounding the device 10 during ozone treatment.

It is to be understood that the present invention is not limited to the embodiments described above, but encompasses any and all embodiments within the scope of the following claims.

I claim:

1. A portable, hand-held corona discharge ozone generator, comprising:
a housing having a plurality of walls enclosing a space;
a fan disposed on said housing and opening into the space;
a plurality of openings disposed in at least one of the walls for providing flow of ambient air through the housing;
means positioned in the space defined by the housing for generating ozone from the ambient air; and
an on/off switch disposed on one of the walls of the housing for activating the means for generating ozone.

2. The portable, hand-held corona discharge ozone generator according to claim 1, wherein said fan is encased in said housing.

3. The portable, hand-held corona discharge ozone generator according to claim 1, wherein one of said walls is a rear wall and wherein said on/off switch is disposed on the rear wall.

4. The portable, hand-held corona discharge ozone generator according to claim 1, wherein said means for generating ozone includes a power module and a ceramic dielectric plate positioned in said housing adjacent said power module.

5. The portable, hand-held corona discharge ozone generator according to claim 1, wherein one of said walls is a bottom wall, the generator further including a plurality of foot members disposed on the bottom wall.

6. The portable, hand-held corona discharge ozone generator according to claim 1, wherein one of said walls is a side wall, the ozone generator further including an AC device disposed on the side wall and further including a power supply module disposed within the housing, the AC device being electrically connected to the power supply module.

7. A portable, hand-held corona discharge ozone generator, comprising:
a housing having a front wall, a rear wall, a top wall, a bottom wall, and two sidewalls walls enclosing a space;
a fan disposed adjacent one of said walls and opening into the space defined by the housing;
openings mounted in the front, rear, bottom and sidewalls to allow circulation of ambient air through the space defined by the housing;
means positioned in the space defined by the housing for generating ozone from the ambient air; and
an on/off switch disposed on the housing for activating the means for generating ozone.

8. The portable, hand-held corona discharge ozone generator according to claim 7, wherein said means for generating ozone includes a power supply module and a ceramic dielectric plate positioned adjacent the power supply module.

9. The portable, hand-held corona discharge ozone generator according to claim 7, further including a plurality of foot members disposed on said bottom wall.

10. The portable, hand-held corona discharge ozone generator according to claim 7, further including an AC device disposed on one of said sidewalls.

11. A portable, hand-held corona discharge ozone generator, comprising:
a housing having a front wall, a rear wall, a top wall, a bottom wall, and two sidewalls walls enclosing a space;
a fan disposed on the housing and opening into the space defined by the housing;
a plurality of openings mounted in the front, rear, bottom and sidewalls to allow circulation of ambient air through the space defined by the housing;
a plurality of foot members for preventing surface marring disposed on the bottom wall of the housing;
an AC device disposed on one of the side walls;
means positioned in the space for generating ozone from ambient air flowing through the housing; and
an on/off switch disposed on one of the housing walls for controlling the means for generating ozone.

12. The portable, hand-held corona discharge ozone generator according to claim 11, wherein said means for generating ozone includes a power supply module and a ceramic dielectric plate positioned adjacent the power supply module.
